(19) United States
(12) Patent Application Publication
(10) Pub. No.: US 2020/0038121 A1
(43) Pub. Date: Feb. 6, 2020

(54) SURGICAL INSTRUMENT, ROBOTIC ARM AND CONTROL SYSTEM FOR A ROBOTIC ARM

(71) Applicant: IMPERIAL INNOVATIONS LIMITED, LONDON (GB)

(72) Inventors: GUANG-ZHONG YANG, LONDON (GB); PIYAMATE WISANUVEJ, LONDON (GB); KONRAD MAREK GÜNTER LEIBRANDT, LONDON (GB); CARLO ALBERTO SENECI, LONDON (GB); JIANZHONG SHANG, LONDON (GB); JINDONG LIU, LONDON (GB)

(21) Appl. No.: 16/303,392

(22) PCT Filed: May 23, 2017
(86) PCT No.: PCT/GB2017/051436

§ 371 Date: Apr. 10, 2019

(30) Foreign Application Priority Data
May 23, 2016 (GB) 1608997.1
May 23, 2016 (GB) 1609002.9
May 23, 2016 (GB) 1609030.0

(51) Int. Cl. A61B 34/30; A61B 18/14; A61B 34/20; A61B 90/00; A61B 90/50

(52) U.S. Cl. CPC A61B 34/30; A61B 18/1445; A61B 2017/00123; A61B 90/06; A61B 90/50; A61B 34/20

(57) ABSTRACT

A surgical instrument comprising: a rigid shaft, at least one elbow joint hingedly coupled to the rigid shaft and a wrist joint coupled to the at least one elbow joint, wherein the wrist joint is configured to provide a first degree of freedom of movement and a second degree of freedom of movement, wherein the second degree of freedom of movement is substantially perpendicular to the first degree of freedom of movement.

### FIELD

The present invention provides a surgical instrument, a robotic arm and a control system for a robotic arm.

### BACKGROUND

Traditional laparoscopic manual instruments are composed of a handle, a rigid shaft and a functional end effector, such as graspers, scissors or suction channels for example. Usually, two laparoscopic instruments are used at the same time by a surgeon. The laparoscopic instruments may be located within a single port or within multiple ports. The common characteristics of all these instruments are that motion is transmitted from the handle to the end effector by exploiting the fulcrum effect between the rigid shaft and the port where the instrument is inserted. Generally, instruments used in laparoscopic surgery provide four degrees of freedom. Taking transanal endoscopic micro-surgery as an example, the workspace available to a surgeon is very limited meaning that manoeuvring the handles of prior art instruments to achieve the fulcrum effect is very challenging and instrument collision is common both at the functional end effector and handle.

Manual articulated laparoscopic surgical tools are inherently bulky and provide challenges to surgeons in terms of safely using such tools within a limited workspace.

A large amount of research has been undertaken into robotic surgical tools for use in many different medical applications. Examples are:

CN104434318 describes an example of a robotic surgical instrument that provides four degrees of freedom.

KR100778387 describes a surgery robot for laparoscopic procedures that comprises a hinged elbow function and a rotatable wrist function.

U.S. Pat. No. 5,624,398 describes an endoscopic robotic surgical tool that provides a shoulder flexion joint, upper arm rotational joint, elbow flexional joint and wrist rotational joint.

U.S. Pat. No. 8,603,135 describes an example of an articulating surgical instrument constructed from a series of links to enable snake-like motion of the surgical instrument.

However, prior art robotic articulated surgical tools are not suitable for use in laparoscopic procedures where space is limited. The prior art robotic articulated surgical tools also do not have sufficient DoF at the tool tip or suitably sized tool tips for use in many laparoscopic procedures.

During surgery, a surgeon is constrained to working within a tightly defined workspace. It is important that the surgeon does not permit surgical instruments to deviate from within the defined workspace or damage or injury could result to a patient. Measures are therefore required to prevent surgical instruments from deviating from the defined workspace.

US2005/0166413 describes a robotic arm that can define a boundary prior to use by moving the arm through a pre-determined set of co-ordinates. In use, if the boundary is crossed the arm is disabled to prevent further movement outside of the boundary.

US2010174410 describes a robotic arm that is operated by depression of a single operating switch.

Robotic surgery typically involves the use of a port device mounted on a robotic arm. The port device comprises a limited number of lumens for receiving respective surgical tools. Often, surgeons utilise all ports in the port device and require additional tools which have to be used independently of the port device.

The present invention seeks to overcome challenges encountered during transanal robotic endoscopic micro-surgery.

### SUMMARY OF THE INVENTION

An aspect of the invention provides a surgical instrument comprising: a rigid shaft, at least one elbow joint hingedly coupled to the rigid shaft and a wrist joint coupled to the at least one elbow joint, wherein the wrist joint is configured to provide a first degree of freedom of movement and a second degree of freedom of movement, wherein the second degree of freedom of movement is substantially perpendicular to the first degree of freedom of movement.

Provision of a surgical instrument with both an elbow joint and a wrist joint is advantageous as this configuration provides a surgeon with at least five degrees of freedom of movement. The rigid shaft transmits linear translation and axial rotation. The at least one elbow joint is connected to the rigid shaft and provides hinged motion between the at least one elbow joint and the wrist joint. The wrist joint provides both hinged and pivoting motion. Such a surgical instrument provides a surgeon with a greater range of motion within a restricted workspace than is possible in the prior art and provides a robotically controlled toolbox having all of the tools used by a surgeon in a conventional manual tool kit for laparoscopic surgery.

In one embodiment, the at least one elbow joint comprises two elbow joints, wherein each elbow joint is arranged to provide a hinged motion in a different direction to the other elbow joint and wherein each elbow joint is movable independently of the other.

In another embodiment, the at least one elbow joint comprises three elbow joints, wherein two of said elbow joints are arranged to provide a hinged motion in the same direction and a third elbow joint is arranged to provide a hinged motion in a different direction to the other elbow joints and wherein each elbow joint is movable independently of the other.

In another embodiment, the at least one elbow joint comprises four elbow joints, wherein a first elbow joint and a second elbow joint are arranged to provide a hinged motion in a first direction and a third elbow joint and a fourth elbow joint are arranged to provide a hinged motion in a different direction to the first elbow joint and the second elbow joint and wherein each elbow joint is movable independently of the other.

Provision of two, three or four elbow joints which provide hinged motion in different directions to one another is beneficial as it confers a further degree of freedom of movement to the surgical instrument. Configuring the surgical instrument such that each elbow joint is movable independently of other elbow joints ensures that each elbow joint is fully decoupled thus simplifying control of the surgical instrument and providing smooth movement of the surgical instrument. Provision of at least six degrees of positioning replicates the human anatomy as far is as possible. This is advantageous as the perceptive experience of the surgeon is made as natural as possible

In another embodiment, the at least one elbow joint comprises a plurality of elbow joints wherein at least two adjacent elbow joints are locked together.

The surgical instrument may further comprise one or more additional elbow joints movable independently of any other elbow joint.

In another embodiment, the surgical instrument further comprises a bipolar or monopolar end effector.

Provision of a bipolar end effector confers a further degree of freedom of movement to the surgical instrument.

Another aspect of the invention provides a surgical instrument comprising: a rigid shaft and at least one elbow joint hingedly connected to the rigid shaft, wherein a primary end effector is connected to the at least one elbow joint and wherein the rigid shaft and the at least one elbow joint define a continuous lumen therethrough, the lumen receiving an auxiliary end effector or providing irrigation or suction functionality.

Combination of an auxiliary tool or suction and/or irrigation functionality with a cutting tool or cauterization tool into a single instrument beneficially reduces the number of tools required during surgery and consequently the number of times that tools require interchanging. Such a combination of tools and/or functionalities also frees up a port on laparoscopic surgical apparatus.

In one embodiment, the primary end effector comprises an electro-cautery knife.

Combination of a monopolar electro-cautery knife with suction and/or irrigation functionality enables a surgeon to cut or cauterize patient tissue and irrigate the surgery site and remove fluid with a single surgical instrument. In the event that a patient bleeds during surgery, a single surgical instrument can be used to efficiently remove fluid and smoke from the surgery site to enable the surgeon to clearly see without the need to exchange tools thus reducing surgery duration and risk to patients.

Another aspect of the invention provides a surgical instrument comprising: a rigid shaft and at least one elbow joint coupled to the rigid shaft, wherein an end effector is coupled to the at least one elbow joint, said end effector being operable by tendons shrouded by Bowden cables arranged between the at least one elbow joint and the end effector to facilitate movement of said end effector relative to the at least one elbow joint.

The use of Bowden cables to manoeuvre the end effector, or end effector, of the surgical instrument enables the length of each tendon controlling the end effector to be approximately equal regardless of orientation of the end effector relative to the at least one elbow joint.

Another aspect of the invention provides a surgical instrument comprising: a rigid shaft and at least one elbow joint coupled to the rigid shaft by way of a mounting arrangement, wherein the mounting arrangement comprises a first part on one of the rigid shaft or elbow joint having a generally circular profile and a second part on the other of the rigid shaft or elbow joint comprising a generally triangular profiled groove for receiving the generally circular profile of the first part therein.

Use of a mounting arrangement comprising a circular projection received within a triangular groove is highly advantageous as such an arrangement reduces the friction in the contact between the two parts of the mounting arrangement, thanks to the single line contact.

Another aspect of the invention provides a protective sleeve for a surgical instrument, the protective sleeve comprising: an elongate flexible sheath having a first end and a second end, wherein the first end comprises an attachment means for attachment of the protective sleeve to a surgical instrument and wherein the second end comprises a closure means.

Use of a protective sleeve prevents contamination of the surgical instrument when not in use and contains bio-hazard materials within the sleeve after use.

In one embodiment, the closure means is a valve or flap.

Use of a valve or flap permits passage of the surgical instrument through the flap or valve during surgery to expose the surgical instrument. When the surgical instrument is withdrawn from a patient after surgery, the valve or flap closes to hygienically stow the surgical instrument within the sleeve.

Another aspect of the invention provides a bipolar end effector comprising: i) a pair of opposed jaws pivotally coupled to permit pivotal motion of one jaw relative to the other, wherein at least one of said opposed jaws comprises a recess for selectively receiving a sensor, and ii) a sensor configured to be secured within said recess.

Another aspect of the invention provides a monopolar end effector comprising: i) an elongate member having a recess for selectively receiving a sensor, and ii) a sensor configured to be secured within said recess.

Another aspect of the invention provides: i) an elongate member having a recess for selectively receiving a sensor, and ii) a sensor configured to be secured within said recess.

The ability to selectively receive a sensor within a recess forming an integral part of a jaw permits the sensor to be replaced each time the end effector is used.

In one embodiment the sensor is a force sensor, temperature sensor, tactile sensor or position sensor.

Another aspect of the invention provides a needle driver comprising a body and a pair of opposed jaws movable between an open position and a closed position, wherein the pair of opposed jaws are biased in the open position by a spring and are closable through use of a tendon to overcome the spring strength when said tendon is tensioned.

In one embodiment each of said pair of opposed jaws is pivotally mounted to the body by way of a respective pin passing through each jaw and received by the body, and wherein each of said respective pins is spaced apart laterally.

Spacing the pins apart laterally provides an enhanced grasping force as compared to prior art end effectors having both pins linearly.

In one embodiment each of said respective pins is positioned adjacent to an edge of the body.

In one embodiment the jaws of the needle driver comprise triangular shaped teeth disposed in alternate rows.

Such an arrangement geometrically locks the section of the needle, preventing its motion. This is a fundamental feature in surgery with flexible instruments where lateral force is required for needle insertion, but where often instruments are not strong enough due to their flexible structure. See attached paper for reference and more information.

In one embodiment the distal end of the needle driver jaws comprises a nose.

The nose may comprise a bulbous end.

The proximal end of the jaws of the needle driver may comprise a disc having a diameter greater than the diameter of the instrument shaft.

The nose beneficially retains a suture thread during knot tying of the suture. The disc prevents the suture from wrapping around the instrument shaft.

In another embodiment the instrument comprises an axial rotational joint proximate the end effector.

The joint allows for about 270° rotation, emulating the human wrist. The instrument structure is changed: the elbow of the instrument is maintained, while the tip of the instrument presents a rotatory joint.

In another embodiment the instrument comprises a pair of jaws operable by a quadrilateral actuation mechanism and biased in a closed position by a return spring.

Another aspect of the invention provides a safety device for a robotic arm comprising a first operation switch and a second operation switch, wherein operation of the robotic arm is effected only by activation of both the first operation switch and the second operation switch.

Laparoscopic surgery is highly complex and requires controlled and accurate movement of surgical tools. Inadvertent movement of a surgical tool could cause damage to a patient. This aspect of the invention seeks to avoid inadvertent movement of surgical tools by requiring a surgeon to consciously operate two operation buttons at the same time to activate the robotic arm.

In one embodiment the first operation switch and second operation switch are arranged so as to be operable by a surgeon using a single hand.

Another aspect of the invention provides a robotic arm comprising a plurality of electromagnetically braked joints and a position sensor associated with each electromagnetically braked joint, wherein each position sensor is operably connected to a processor, said processor monitoring the position of each electromagnetically braked joint relative to a pre-determined spatial threshold and locking each of said electromagnetically braked joints upon the processor detecting a signal from one or more position sensors signifying approach of one or more of said electromagnetically braked joints to a spatial threshold.

Surgeons undertaking laparascopic surgery are required to work within a tightly defined workspace. Positioning of surgical tools outside of the defined workspace is undesirable and could lead to damage to a patient. To prevent unwanted positioning of surgical tools, the robotic arm is provided with a lockout mechanism to prevent further movement of the robotic arm when a proximity sensor detects that a surgical tool has exited the defined workspace.

In one embodiment, the robotic arm further comprises a rotary encoder for identifying the position of a surgical tool relative to a defined workspace.

In another embodiment the lockout mechanism permits movement of the surgical tool in a reverse manner from the point of lockout using the rotary encoder to mimic prior movement of the surgical tool in reverse until the surgical tool achieves its original position prior to commencement of surgery.

Once the movement of the robotic arm has been locked it is important that the surgeon is able to take steps to move the surgical tool back within the defined work area while preventing further movement of the surgical tool outside of the defined work area. Use of the rotary encoder provides full details of all movement of the robotic arm during a surgery such that the robotic arm can be moved in reverse to bring the surgical tool back into the defined work area using data gathered by the rotary encoder.

Another aspect of the invention provides a method of determining a force characteristic comprising: i) providing a robotic arm that comprises a plurality of electromagnetically braked joints, wherein each joint is driven by drive means, a rotary encoder, and an end effector; ii) establishing a base line force characteristic when each electromagnetically braked joint is activated; iii) measuring rotation of the end effector using the rotary encoder; determining the stiffness characteristics of each drive means; and iv) determining a force characteristic for the end effector from a torque applied to each electromagnetic joint.

Another aspect of the invention provides a joint for a robotic arm comprising an electromagnetically braked joint and a backlash-free differential drive.

The combination of an electromagnetic brake with a backlash-free differential drive has the advantage of small footprint and large output torque comparing to the conventional solutions: 1. combination of a motor and a differential drive, in which the motor has much smaller holding torque comparing to the same size brake; 2. only using brake without differential drive, in which the output torque is less and the footprint is larger than our solution.

Another aspect of the invention provides a control system for a robotic surgical system comprising a plurality of motor controllers, a safety watchdog module, and a motherboard, wherein the safety watchdog and plurality of motor controllers are operably connected to the motherboard and wherein the safety watchdog module monitors at least one parameter of the robotic surgical system and is configured to isolate power from the motor controllers in response to detection by the safety watchdog module of one or parameters deviating from a pre-determined range or exceeding a pre-determined threshold.

Provision of a safety watchdog beneficially reduces the risk of erroneous operation of a robotic surgical instrument and minimises risk of injury or damage to a patient.

In one embodiment the safety watchdog module and plurality of motor controllers are modular components of the motherboard and can be selectively removed and replaced without removal of other modular components of the motherboard.

The use of modular components reduces the footprint of the robot control system as compared to the prior art and generally increases and optimises the ability to service and upgrade the robot control system.

In one embodiment the plurality of motor controller modules comprise four motor controller modules, wherein each motor controller module is configured to be operably coupled to up to two motors.

In one embodiment each motor controller module has a unique identifier.

In one embodiment the motherboard has an associated address changeable through operation of one or more switching means.

The ability to change the address of the motherboard enables the address of the entire robot control system to be changed to enable more than one robot control system to be operably coupled to a computer system.

### FIGURES

The invention will now be described by way of reference to the following figures:

FIG. 1 shows a surgical instrument according to aspects of the invention;

FIG. 2 shows a first and second section of the surgical instrument of FIG. 1;

FIG. 3 shows an illustrative view of the degrees of freedom of movement of the surgical instrument of FIG. 1;

FIG. 4 shows a further view of the surgical instrument of FIG. 1;

FIG. 5 shows a PTFE catheter for use with embodiments of the invention;

FIG. 6 shows an example surgical instrument combining a primary end effector (bipolar) and suction and/or irrigation functionality;

FIG. 7 shows an instrument base for coupling a surgical instrument to a robotic arm assembly;

FIG. 8 shows a robotic arm according to aspects of the invention;

FIG. 9 shows a schematic of a control system for robotic systems;

FIG. 10 shows a view of a protective sleeve for use with embodiments of the invention;

FIG. 11 shows a detailed view of the protective sleeve of FIG. 10;

FIG. 12 shows a view of an end effector adapted to receive a sensor therein;

FIG. 13 shows a first view of a needle driver end effector;

FIG. 14 shows a second view of the needle driver of FIG. 13;

FIG. 15 shows an alternative embodiment of a needle driver;

FIG. 16 shows a side view of an end effector with axial rotation imparted at the end effector.

### DESCRIPTION

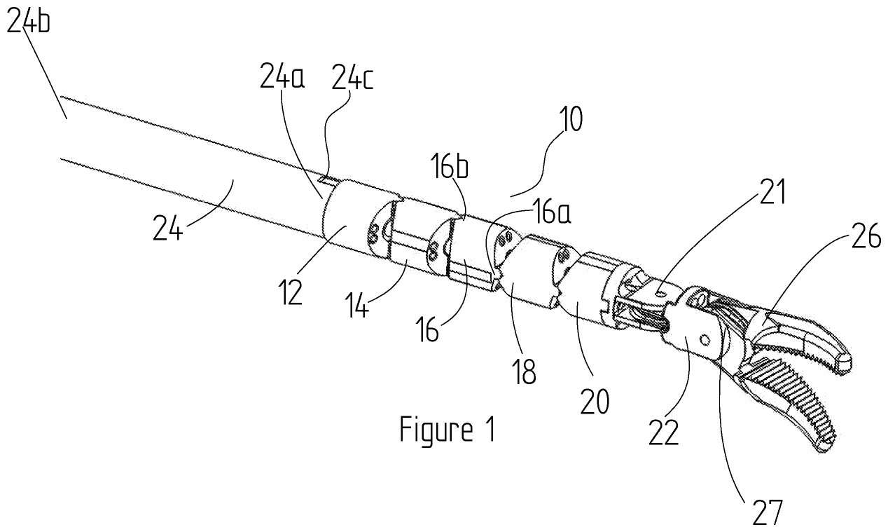

Surgical instruments according to aspects of the invention are illustrated generally in FIG. 1. A surgical instrument (10) comprises a plurality of sections (12, 14, 16, 18, 20, 22) connected to a rigid shaft (24). The rigid shaft (24) is connected to an instrument base (not shown in FIG. 1). An instrument tip (26), also referred to as an end-effector herein, is connected to the section (22) furthest away from the rigid shaft (24).

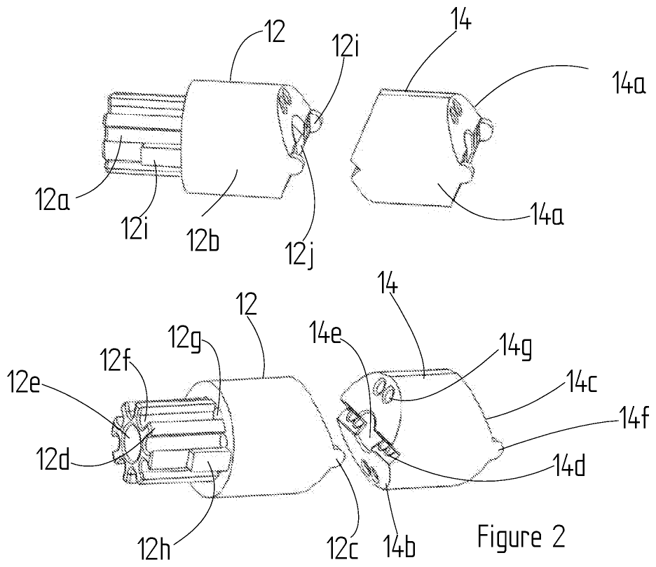

A first section (12), as illustrated in FIG. 2, is fixedly connected to the rigid shaft (24) by way of a splined connection (12a). The first section (12) comprises a generally cylindrical body (12b) having the splined connection (12a) at one end thereof and a mounting feature (12c) at the other end thereof. The splined connection (12a) is 4 mm long and comprises eight projections (12d) extending radially from a central lumen (12e). Each of the eight projections (12d) are evenly spaced apart with a length of 1.7 mm measured from the central axis of the first section (12) and define a scallop (12f) between each adjacent pair of the eight projections (12d). Each scallop (12f) receives a tendon (not shown in FIGS. 2a and 2b) with each tendon passing through the generally cylindrical body (12b) of the first section (12) through a respective hole (12g) arranged around the central lumen (12e). The splined connection (12a) further comprises a locking formation (12h) for restricting or preventing rotation of the first section (12) relative to the rigid shaft (24).

The central lumen (12e) has a cylindrical profile and an internal diameter of between 1.5 mm and 3 mm.

The mounting feature (12c) comprises an opposite pair of generally semi-circular tabs (12i) extending longitudinally away from the generally cylindrical body (12b). Each generally semi-circular tab (12i) has a radius of 0.5 m and a thickness of between 0.5 mm and 1.5 mm. The generally semi-circular tabs (12i) are mounted at the extreme end of the body (12b) and define between them a flattened apex (12j) from which the generally cylindrical body (12b) is chamfered in both directions away from the end of the first section (12) to enable relative movement of an adjacent section (14). The angle of chamfer in each direction is ninety four degrees to enable the adjacent section (14) to hingedly rotate through eighty degrees relative to the first section (12).

The rigid shaft (24), as shown in FIG. 1, comprises a hollow tube having an outer diameter of 5 mm and an inner diameter of 4 mm. The rigid shaft (24) is formed from stainless steel and is between 200 mm and 300 mm long. The first end (24a) of the rigid shaft (24) is configured to receive the splined connection (12a) of the first section (12) and restrict rotation of the splined connection (12a) of the first section (12) therein. The rigid shaft (24) is connected at the second end (12b) thereof to an instrument base (not shown in FIG. 1 or FIG. 2). The rigid shaft (24) is used to transmit linear translation and axial rotation motion from the instrument base to the end effector (26). All other degrees of freedom are controlled through use of the tendons that pass through the rigid shaft (24) to the surgical instrument sections (12, 14, 16, 18, 20, 22).

The rigid shaft (24) further comprises a complimentary locking formation (24c) for cooperation with locking formation (12h) of the first section (12) to prevent rotation of the first section (12) relative to the rigid shaft (24).

The second section (14), as illustrated in FIG. 2, is hingedly connected to the first section (12). The second section (14) comprises a generally cylindrical body (14a) having a first end (14b) and a second end (14c). The first end (14b) of the second section (14) comprises a groove of triangular cross section (14d) for receiving the generally semi-circular tabs (12i) of the mounting formation (12c) of the first section (12). The profile of the cylindrical body (14a) of the second section (14) is chamfered away from the triangular groove (14d) in both directions towards the second end (14c). The angle of chamfer in each direction is ninety four degrees to enable relative hinged movement between the first section (12) and the second section (14). The second section (14) further comprises an internal lumen (14e) substantially similar to the internal lumen (12e) of the first section (12).

The second end (14c) of the second section (14) comprises a mounting feature (14f) substantially the same as the mounting feature (12c) of the first section (12). A plurality of holes (14g) for receiving respective tendons pass longitudinally through the cylindrical body (14a) and surround the lumen (14e).

The third and fourth sections (16, 18) are substantially the same as the second section (14) and connected together in a snake like formation. The sections (12, 14, 16, 18) can be arranged to provide hinged movement in any direction as necessary according to intended use of the surgical instrument (10). The second section (14) as illustrated in FIG. 2 shows the mounting formation (14e) and triangular groove (14d) aligned. In other embodiments, such as illustrated in FIG. 1, the mounting formation (16a) and triangular groove (16b) are orientated at ninety degrees from one another. It will be appreciated that the orientation of the mounting formation (16a) and triangular groove (16b) can be selected based on the range of motion required for a particular application.

In some embodiments, each of the second, third and fourth sections (14, 16, 18) are movable independently of one another to provide maximum dexterity. Other embodiments require less dexterity and two or more adjacent sections may be locked together causing such sections to move in unison.

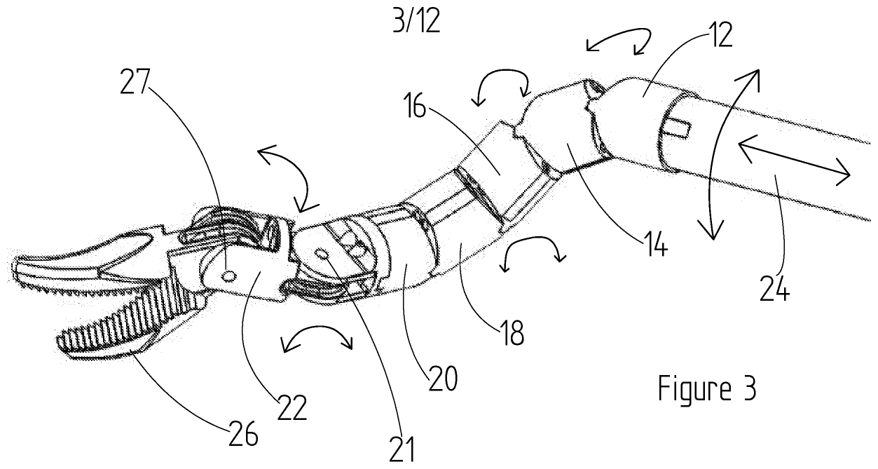

FIG. 3 illustrates the degrees of freedom of movement of a surgical instrument (10) according to aspects of the invention. The arrows shown indicate the general direction of movement of each component of the surgical instrument (10).

In one embodiment the rigid shaft (24) imparts translational movement and axial rotation to the surgical instrument (10). None of the sections (12, 14, 16, 18, 20, 22) or end effector (26) have the independent ability to translate or rotate around the axis of the surgical instrument (10). The first section (12) is positionally fixed relative to the rigid shaft (24). The second section (14) defines an elbow joint with the first section (12) and is hingedly movable relative to the first section (12) through an angular range of movement of eighty degrees. The third section (16) defines an elbow joint with the second section (14) and is hingedly movable relative to the second section (14) through an angular range of movement of eighty degrees. The fourth section (18) defines an elbow joint with the third section and is hingedly movable relative to the third section (16) through an angular range of movement of up to eighty degrees. In some embodiments the angular range of movement is sixty degrees.

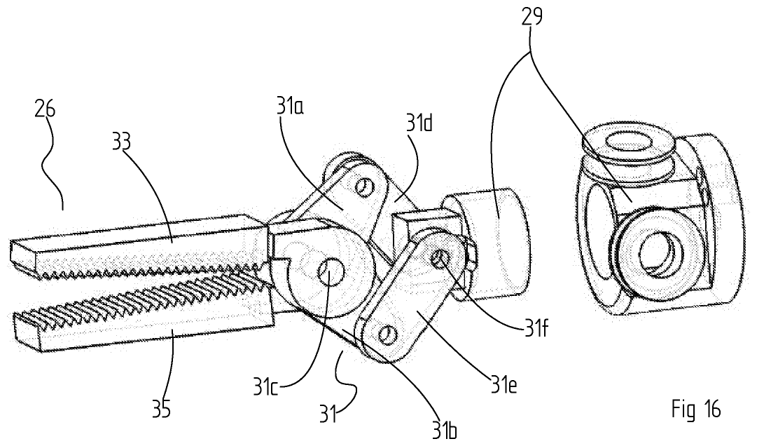

In another embodiment axial rotation is imparted into the end effector (26) by an axial rotational joint (29), as shown in FIG. 16, The axial rotational joint (29) allows for two hundred and seventy degree axial rotation of the end effector (26). Axial rotation is imparted by way of a pair of tendons (not shown).

As illustrated in FIG. 16, an instrument comprising an axial rotational joint (29) adjacent to or integral with the end effector (26) further comprises a quadrilateral actuation mechanism (31) for opening and closing the jaws (33, 35). The quadrilateral mechanism comprises first and second arms (31a, 31b) connected to each of the jaws (33, 35) by a common pivot point (31c) and third and fourth arms (31d, 31e) respectively pivotally connected to the first and second arms (31a, 31b) and at a common pivot point (31f) acting as an anchor point for a drive tendon (31g). The drive tendon (31g) is operatively connected to a return spring (not shown) such that the jaws (33, 35) are biased in a closed configuration by the return spring.

The fifth section (20) and sixth section together define part of the wrist joint of the surgical instrument (10). The fifth section (20) defines an elbow with the fourth section (18) and is hingedly movable relative to the fourth section (18). The fifth section (20) also defines a separate hinged joint (21) with the sixth section (22). The sixth section (22) is hingedly movable relative to the fifth section (20). The sixth section (22) defines a hinged connection (27) with an end effector (26) which is arranged perpendicular to the hinged connection between the fifth section (20) and sixth section (22). The hinged connection (27) between the sixth section (22) and end effector (26) and the hinged connection (21) between the fifth section (20) and sixth section (22) together define all DoF provided by the wrist joint.

In some embodiments, each of the sections (14, 16, 18, 20, 22) is independently movable relative to adjacent sections (14, 16, 18, 20, 22) This arrangement enables the surgical instrument (10) to be manoeuvred in a snake like manner to provide an optimised motion path for a surgeon during surgery. In other embodiments sections (14, 16, 18, 20) may be coupled to adjacent sections (12, 14, 16, 18, 20) such that one or more adjacent sections (14, 16, 18, 20) move together.

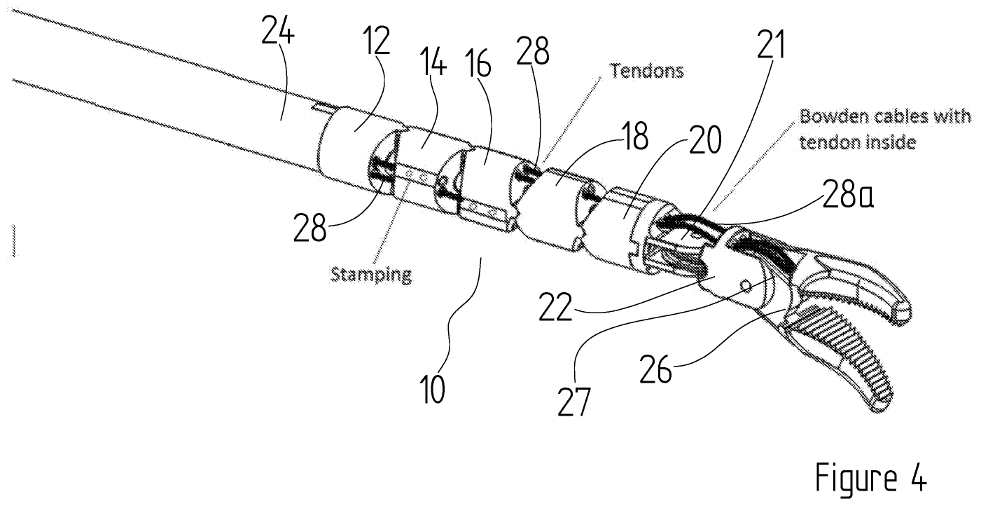

As shown in FIG. 4, tendons (28) passing through the lumen in the rigid shaft (24) and through respective holes in each section (12, 14, 16, 18, 20, 22) and end effector (26) are used to provide independent control to each respective section (12, 14, 16, 18, 20, 22) and end effector (26). Each section (12, 14, 16, 18, 20, 22) and end effector (26) is associated with an antagonistic pair of tendons (28). By antagonistic it is meant that tensioning one of the pair of antagonistic tendons will result in movement of a section (12, 14, 16, 18, 20, 22) or end effector (26) in one direction and tensioning of the other of the pair of antagonistic tendons will result in movement of a section (12, 14, 16, 18, 20, 22) or end effector (26) in the other direction.

Each one of a pair of antagonistic tendons (28) is terminated at a section (14, 16, 18, 20, 22) or end effector (26). Termination of tendons (28) is effected by collapsing the tendon holes (12g—for the first section) through the relevant section (14, 16, 18, 20, 22) or end effector (26) to prevent further movement of the tendons (28) relative to that section (14, 16, 18, 20, 22) or end effector (26).

Tendons (28) associated with control of sections (18, 20, 22) located nearer to the end effector (26) pass through the neutral axis of the bending plane of adjacent sections to reduce motion coupling between adjacent sections.

In some embodiments only a selected number of sections are required to be independently controlled. In such embodiments, tendons (28) provide passive control to those sections not associated with a pair of terminated antagonistic tendons (28). Such an embodiment might be used in a surgical instrument used for cutting tissue where high manual dexterity is not needed. Surgical instruments used for manipulating tissue or using a needle and thread need a greater degree of manual dexterity.

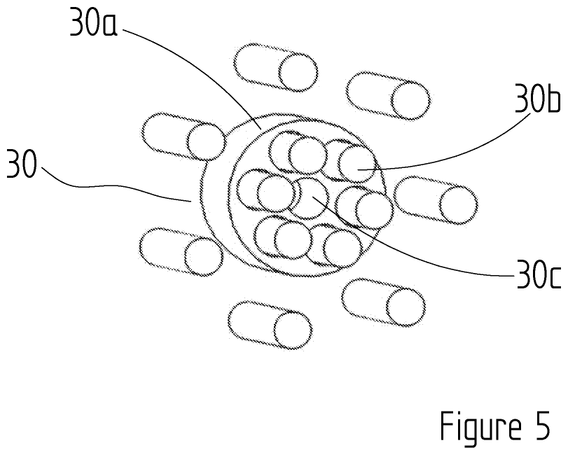

The lumens (12e, 14e, for example) in each section in some embodiments are fitted with a multi-lumen polytetrafluoroethylene (PTFE) catheter (30) as shown in FIG. 5. The PTFE catheter (30) comprises a generally cylindrical rod (30a) having a plurality of lumens (30b) therethrough surrounding a central lumen (30c). Each of the plurality of lumens (30b) is configured to receive a tendon for independent control of the end effector (26).

The PTFE catheter (30) assists in keeping the tendons for controlling the end effector passing therethrough as close as possible to the bending axis of the surgical instrument (10) to prevent a joint coupling effect between adjacent hingedly connected components of the surgical instrument (10). The PTFE catheter (30) additionally assists to reduce friction between adjacent tendons (28) and between tendons (28) and elbow joints.

The tendons for the end effector (26) are shrouded by Bowden cables (28a), i.e. a flexible cable used to transmit mechanical force or energy by the movement of an inner cable relative to a hollow outer cable housing. The PTFE catheter (30) is only needed if the end effector (26) comprises an articulated tool providing a further degree of freedom of positioning such as a grasper or scissors.

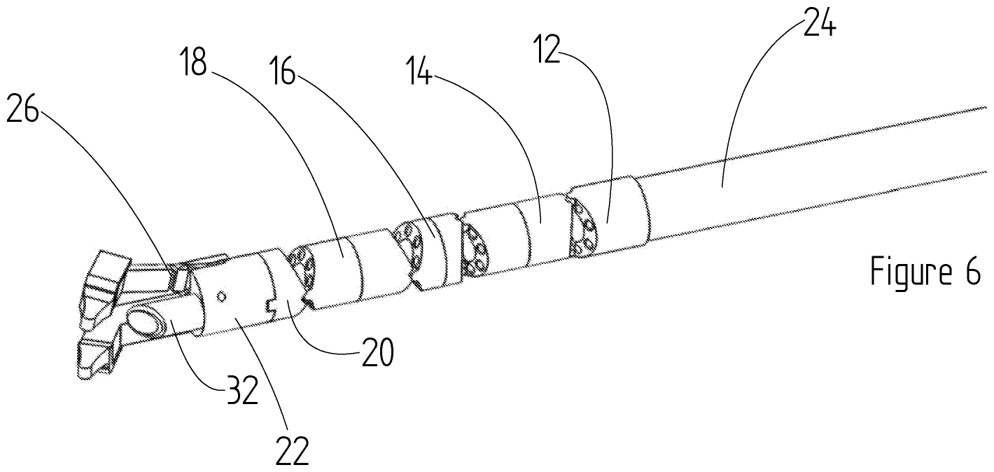

In place of the PTFE catheter (30), the lumen (12e, 14e, for example) through each of the elbow joints can receive a flexible suction and/or irrigation tube (32) as shown in FIG. 6. The flexible tube (32) is intended for use with either a monopolar knife or bipolar tweezers. Both types of tool require electricity to be supplied to the tip of the end effector (26). In the case of a monopolar tool, electricity is conveyed to the tip of the end effector (26) through the metal structure of the end effector (26). In the case of bipolar tweezers, electricity is conveyed to one side of the tweezers by the metal structure of the end effector (26). An electrical wire conveys electricity from the electrified tweezer side to the other tweezer side which is otherwise electrically isolated from the first side.

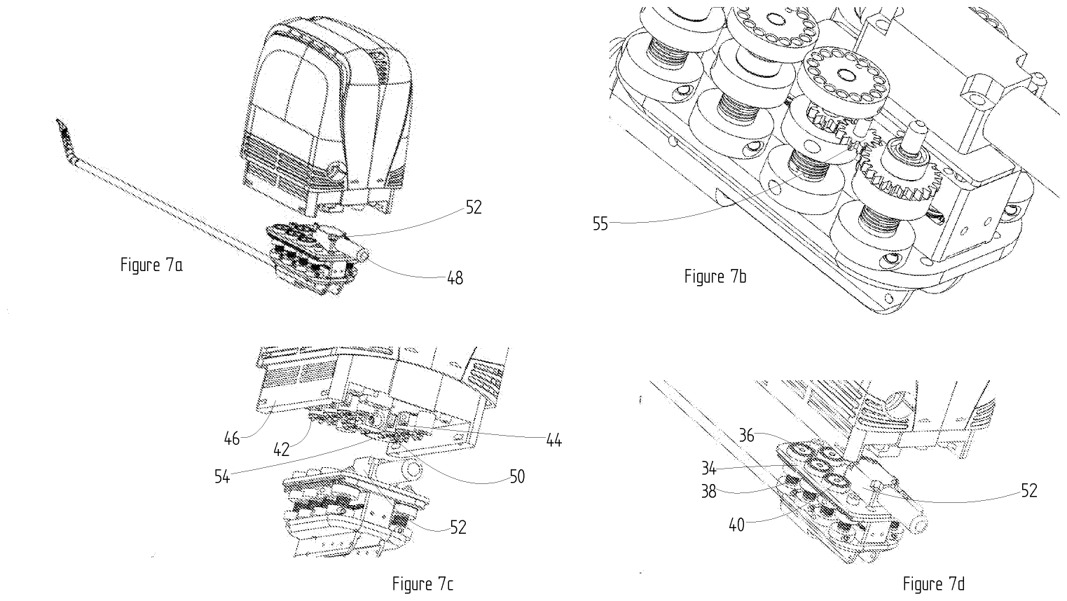

The instrument base (34), as shown in FIGS. 7a to 7d, comprises six motor couplings (36) each associated with respective capstans (38) around which individual tendons (28) are wound. Each motor coupling (36) on the instrument base (34) comprises a plurality of holes (40) for engagement with a plurality of corresponding pins (42) on a corresponding motor coupling (44) on a motor pack (46). Each motor coupling (44) on the motor pack (46) is associated with a respective independently driven motor. Each motor coupling (36) on the instrument base (34) is made from medical grade polyetheretherketon.

Upon attaching the instrument base (34) to the motor pack (46), the motor couplings (36) on the instrument base (34) are each coupled to respective corresponding motor couplings (44) on the motor pack (46) by rotating the motor couplings (36, 44) on either the instrument base (34) or motor pack (44) until the pins (42) on the motor couplings (44) on the motor coupling (46) engage with the holes (40) on the motor couplings (36) on the instrument base (34). Either or both of the motor couplings (36, 44) on the instrument base (34) and/or motor pack (46) are spring loaded to provide a positive engagement between the pins (42) on the motor couplings (44) on the motor pack (46) and the holes (40) on the motor couplings (36) on the instrument base (34). The instrument base (34) is secured to the motor pack (46) by inserting a locking pin (48) through a locking feature (50) on the motor pack (46) and into a corresponding locking feature (52) on the instrument base (34).

Each motor coupling (36) on the instrument base (34) is associated with driving a capstan (38) to wind a tendon (28) for operating a section (12, 14, 16, 18, 20, 22) or end effector (26). An idle gear (55) (as shown in FIG. 7b) is positioned between two capstans. A gear ratio of 2:1 to between the two capstans reflect the tendon travel difference between the two parallel joints to enable a single motor to drive the two capstans to achieve the desired actuation of the two parallel joints between sections (12, 14, 16). The joints between sections (16, 18, 20) are coupled in the same way by another idle gear on the other side of the instrument base (34).

A translation gear (54) is attached to a motor output shaft directly. The gear (54) drives the instrument and motor pack moving along a rack (not shown) for linear translation.

The end effector (26) can be a grasper, needle driver or scissors, for example and is coupled to the final section (20) of the surgical instrument (10) by way of an end effector (22). The end effector (26) is coupled to the final section (22) of the surgical instrument (10) by way of a hinge arrangement orientated perpendicularly to the hinged coupling between the fourth section (18) and final section (22). The hinged coupling between the final section (22) and end effector (26) is also perpendicular to the hinged coupling between the fifth section (20) and sixth section (22).

Examples of end effector (26) described by aspects of the invention include: i) a wristed grasper—seven degrees of freedom tool with grasper jaws which can be either straight or curved and which is used to manipulate tissue, ii) wristed scissors—seven degrees of freedom wristed tool with scissor blades used to cut tissue with either curved or straight blades, iii) non-wristed scissors—six degrees of freedom tool with scissor blades used to cut tissue with either curved or straight blades, iv) wristed needle driver—seven degrees of freedom tool with straight short jaws having a diamond shaped knurling to grip onto surgical needles, v) non-wristed needle driver—six degrees of freedom tool with straight short jaws having a diamond shaped knurling to grip onto surgical needles, vi) monopolar knife with suction/irrigation—a four degree of freedom multi-functional tool without wrist joint and jaws used for tissue re-section, tissue cauterization, suction of liquid/smoke and irrigation, vii) Bipolar tweezers with suction/irrigation—a five degree of freedom non-wristed multifunctional tool having one moving jaw and used for tissue resection, tissue cauterization, suction of liquid/smoke and irrigation, viii) non-wristed grasper tools.

A monopolar tool, i.e. a knife, can provide electrocautery (tissue cut and cauterization) as well as suction and irrigation. Such a tool is multi-functional and enables a surgeon to excise and cauterise tissue while at the same time removing smoke by way of the suction function. Irrigation is used to wash the wound and suction can again be used to clear the wound from fluids, i.e. blood and saline.

A particular example of end effector (26) is a jawed grasper (400) having a pair of opposed jaws. Each jaw (400a) of the end effector (26) is formed of unitary construction and comprises a gripping surface (400b) defined by the internal surface of an elongate member (400c). The elongate member (400c) further comprises a recess (400d) opposite the gripping surface (400b). The recess (400d) extends longitudinally along the elongate member (400c) and is configured to receive a sensor (402) shaped to correspond with the overall profile of the elongate member (400c). The elongate member (400c) is joined to a mounting boss (400e) defined by two spaced apart plates (400f, 400g) having a gap therebetween. A mounting hole (400h) passes through the mounting boss (400e) for receiving a pivot (not shown).

The sensor (402) has a first insertion portion (402a) and a second insertion portion (402b) which are cooperable with a respective first receiving portion (400i) and second receiving portion (400j) of the elongate member (400c) of the jaw (400a). The sensor (402) can be a force sensor, temperature sensor, tactile sensor, for example.

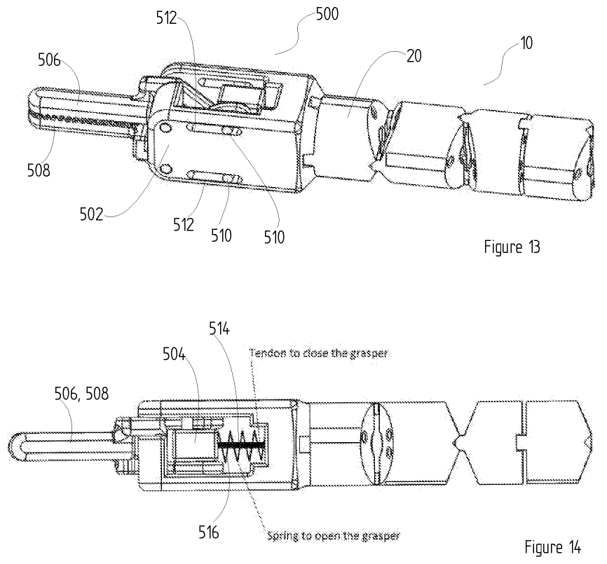

Another example of end effector (26) is a needle driver (500) as illustrated in FIGS. 13 and 14. The needle driver (500) is fixedly coupled to the final section (20) of the surgical instrument (10) by way of a splined connection (20a). The needle driver (500) comprises a body (502) having a mounting arrangement (504) co-operable with each of a pair of opposed grasping jaws (506, 508). The mounting arrangement (504) facilitates pivotal movement of a mounting part of each jaw (506, 508) to permit the jaws (506, 508) to open and close by way of a pin (510) passing through each jaw (506, 508) and the body (502). As shown in FIG. 13, there are two pins (510), one for each jaw (506, 508), which are spaced apart laterally and positioned adjacent the edge of the body (502) and terminate in a groove (512) on each of opposing sides of the body (502).

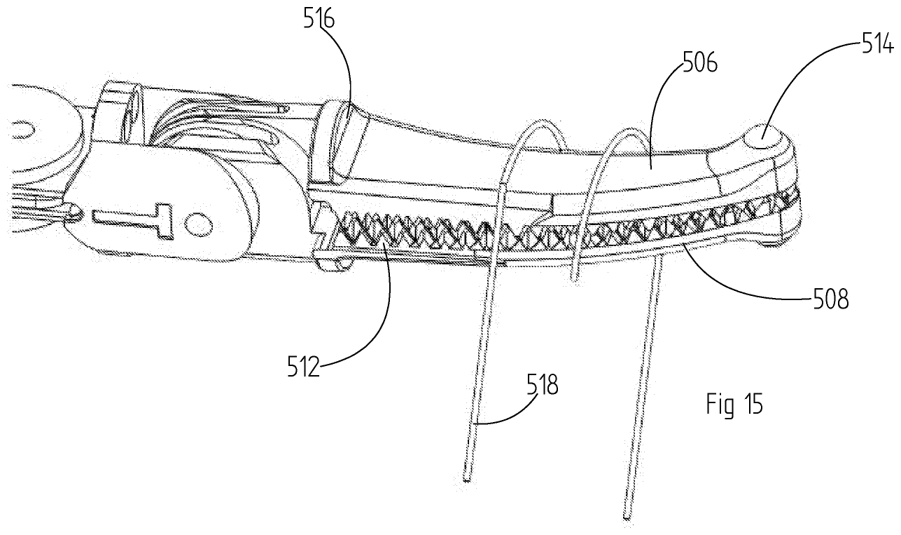

The teeth (512), as better illustrated in FIG. 15, are triangular shaped and disposed in alternate rows to permit interlocking of the teeth (512) when the jaws (506, 508) are closed. Each tooth (512) has a base that measures 0.25 mm, a height of 0.5 mm and a width of 0.35 mm. The teeth are placed in rows spaced 0.47 mm apart. Every row of teeth presents five teeth. Alternating the position of the teeth ensures that the teeth from a first jaw (506) fall between spaced between neighbouring teeth (512) on the second jaw (508). Furthermore, the tip of the needle driver (500) features a nose (514) that is used to retain the thread of a suture (518) during knot tying thus preventing escape of the suture from the jaws (506, 508). The nose (514) comprises a bulbous end at the distal end of each jaw (506, 508). The proximal end of the jaws (506, 508) features a disc (516) having an outer diameter greater than the diameter of the instrument shaft. The disc (516) prevents the suture thread from wrapping around the instrument shaft. The profile of the jaws (506, 508) is rounded in some embodiments.

Movement of the jaws (506, 508) is controlled by a tendon (514) and a spring (516). The jaws (506, 508) are biased in an open position by the spring (516). The spring (514) tension is overcome by tensioning the tendon (514) to close the jaws (506, 508).

The motor pack (46) is selectively mountable to a robotic arm (100) or to a port as described in further detail below.

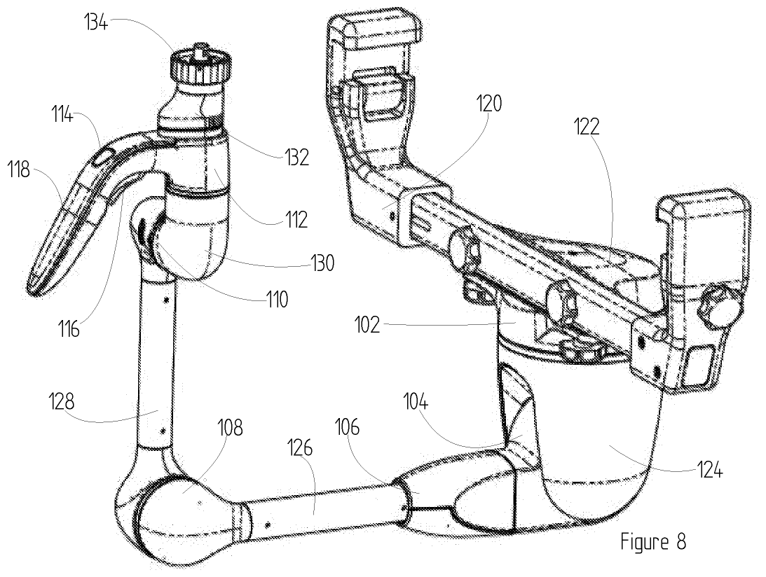

The robotic arm (100), as shown in FIG. 8, comprises six electromagnetically braked joints (102, 104, 106, 108, 110, 112). Each electromagnetically braked joint (102, 104, 106, 108, 110, 112) comprises an electromagnetic brake and a backlash-free differential drive equipped with an absolute angle joint encoder. The electromagnetic brakes are biased in an on position and releasable by depression of two operation switches (114, 116) located on a handle (118). The robotic arm (100) is mountable to a hospital bed by way of a mounting formation (120) coupled to the robotic arm (100).

The mounting formation (120) is coupled to an anchor (122). The anchor (122) is coupled to a shoulder (124) by way of a first electromagnetically braked joint (102). The anchor (122) provides horizontal rotation relative to the shoulder (124). The shoulder (124) is coupled to a horizontal shaft (126) by way of a second electromagnetically braked joint (104). The shoulder (124) provides pivotal rotation relative to the horizontal shaft (126) in the direction of the longitudinal axis of the horizontal shaft (126). The horizontal shaft (126) extends through a third electromagnetically braked joint (106). The horizontal shaft (126) provides rotational positioning relative to the shoulder (124). The opposite end of the horizontal shaft (126) is coupled to a fourth electromagnetically braked joint (108). The fourth electromagnetically braked joint (108) is coupled to a vertical shaft (128). The vertical shaft (128) provides rotational positioning relative to the horizontal shaft (126). The vertical shaft (128) is coupled at the other end to a fifth electromagnetically braked joint (110). The fifth electromagnetically braked joint (110) is coupled to an elbow (130). The elbow (130) provides rotational positioning around a horizontal axis parallel to the horizontal axis of the horizontal shaft (126). The elbow (130) is coupled to a sixth electromagnetically braked joint (112) at the other end thereof. The sixth electromagnetically braked joint (112) is coupled to the handle (118). The handle is free to rotate around a vertical axis in order to position an adaptor (132) coupled to the handle (118).

The adaptor (132) mounts the motor pack (46) and consequently the surgical instrument (10) to the robotic arm (100).

In use, the robotic arm (100) is mounted to a standard operating table by way of the mounting formation (120) which clamps the robotic arm (100) to the side rails of the standard operating table. The robotic arm (100) and surgical instrument (10) are both electrically powered from a mains supply power outlet through an AC/DC power adaptor. The power supply controls each of the electromagnetically braked joints (102, 104, 106, 108, 110, 112) with electromagnets associated with each being locked in place unless the operation switches (114, 116) on the handle (118) are depressed. Upon depression of both operation switches (114, 116) on the handle (118), all electromagnets are released permitted an operator to manoeuvre the robotic arm (100) through all six electromagnetically braked joints (102, 104, 106, 108, 110, 112). Once the robotic arm (100) is in the desired position the operating switches (114, 116) on the handle (118) are released by the operator and all electromagnets are applied to lock all six electromagnetically braked joints (102, 104, 106, 108, 110, 112). The electromagnets will only be released if both operation switches (114, 116) on the handle (118) are depressed. If only one operation switch (114, 116) is depressed, none of the electromagnets will be released and the operator will not be able to manoeuvre the robotic arm (100) through any of the electromagnetically braked joints (102, 104, 106, 108, 110, 112). This is a safety feature to prevent inadvertent movement of the robotic arm (100).

When the whole arm is locked, the differential driver's output shaft will have a trivial relative rotation to the driver's body if a force is applied to the arm's end-effector. Such rotation can be measured by the joint angle encoder and consequently the torque on the differential drive caused by the force on the end-effector can be calculated considering the stiffness of the differential drive. By taking into account the torque on every joint, the magnitude and direction of the force on the end-effector can be calculated. The combination of an electromagnetic brake with a backlash-free differential drive has the advantage of small footprint and large output torque comparing to the conventional solutions: 1. combination of a motor and a differential drive, in which the motor has much smaller holding torque comparing to the same size brake; 2. only using brake without differential drive, in which the output torque is less and the footprint is larger than our solution.

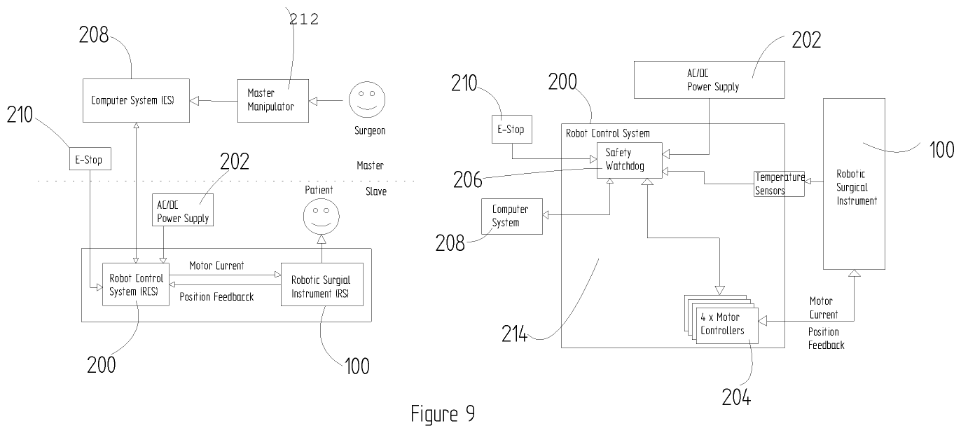

Once a motor pack (46) is mounted to the adaptor (132) and a surgical instrument (10) is coupled to the motor pack (46), power is applied to the motor pack by way of a mains power supply. The motor pack (46) is controlled by a robot control system (200) as illustrated in FIG. 9.

The robot control system (200) is powered by a separate mains power supply (202) and comprises a plurality of motor controller modules (204), four are shown in FIG. 9, and a safety watchdog module (206). The safety watchdog (206) is connected between the mains power supply (202) and the plurality of motor controller modules (204). The robot control system (200) is connected between the robotic surgical instrument (100) and a computer system (208). The robot control system (200) is further provided with an emergency stop button (210) for cutting all power to the robot control system (200) and thus the surgical instrument (10). A master manipulator (212) is connected to the computer system (208). The computer system (208) interprets movement of the master manipulator (212) to determine the desired action of the surgical instrument (10) and sends appropriate instructions to the robot control system (200) via a RS-485 bus to drive the plurality of motor controllers (204).

The safety watchdog module (206) monitors a number of parameters of the robot control system (200) and/or surgical instrument (10) such as temperature and motor current for example. If the safety watchdog module (206) detects that a parameter has deviated from a pre-determined range or exceeded a pre-determined threshold, the safety watchdog module (206) will cut all power to the motor controller modules (204) to prevent erroneous operation and/or damage/injury to a patient. The safety watchdog module (206) also listens to communication between the computer system (208) and robot control system (200) and between the robot control system (200) and the surgical instrument (10). If instructions are detected that fall outside of accepted operating parameters the safety watchdog module (206) will cut all power to the robot control system (200) to prevent erroneous operation and/or damage/injury to a patient.

The safety watchdog module (206) is a modular component that plugs into a motherboard (214). Each motor controller module (204) is also a modular component that plugs into the motherboard (214). Each motor controller module (204) can control up to two motors and the motherboard (214) can accommodate up to four motor controller modules (204) allowing connection of up to eight motors for driving the robotic surgical instrument (100). This disclosure is not intended to be limiting; other embodiments may be capable of accommodating further motor control modules and each motor control module may be capable of controlling one, two or more motors.

The adaptor (132) includes an electrical connector (134) which can supply power and control signals via the internal wiring of the robotic arm (100). The motor pack (46) can be powered and controlled via either the electrical connection (134) or independent cables.

To ensure that the surgical instrument (10) is only movable within a pre-defined boundary, a three dimensional boundary space or spatial threshold is defined prior to commencing surgery. The three dimensional boundary space is defined by moving the robotic arm (100) through a series of spatial points and recording each spatial point as a boundary point. The robotic arm during surgery is only permitted to move within the three dimensional boundary and is automatically locked should it hit, or in some instances approach, the three dimensional boundary.

Once movement of the robotic arm (100) is locked, there are a number of ways that it can be unlocked to resume surgery. Two examples will now be described.

In a first example, the robotic arm (100) comprises a rotary encoder that monitors every movement of each of the electromagnetically braked joints (102, 104, 106, 108, 112) and the surgical instrument end effector (22). Each movement is recorded as a data point relative to a respective origin point. The rotary encoder permits each electromagnetically braked joint (102, 104, 106, 108, 110, 112) and thus the surgical instrument end effector (22) to move through each data point in reverse. Once each data point is determined as being equal to a respective origin point, each of the electromagnetically braked joints (102, 104, 106, 108, 110, 112) is fully released.

In a second example, force detection means are associated with each of the electromagnetically braked joints (102, 104, 106, 108, 110, 112). A processor equates a force applied by a surgeon to a master manipulator (212) to direction and unlocks the electromagnetically braked joints (102, 104, 106, 108, 110, 112) if it is determined that all of the electro magnetically braked joints (102, 104, 106, 108, 110, 112) and the surgical instrument end effector (22) would be moved away from the three-dimensional boundary. If it is determined that one or more of the electromagnetically braked joints (102, 104, 106, 108, 110, 112) and/or the end effector (22) would be moved towards or cross the three-dimensional boundary, each of the electromagnetically braked joints (102, 104, 106, 108, 110, 112) would remain locked and movement would be resisted.

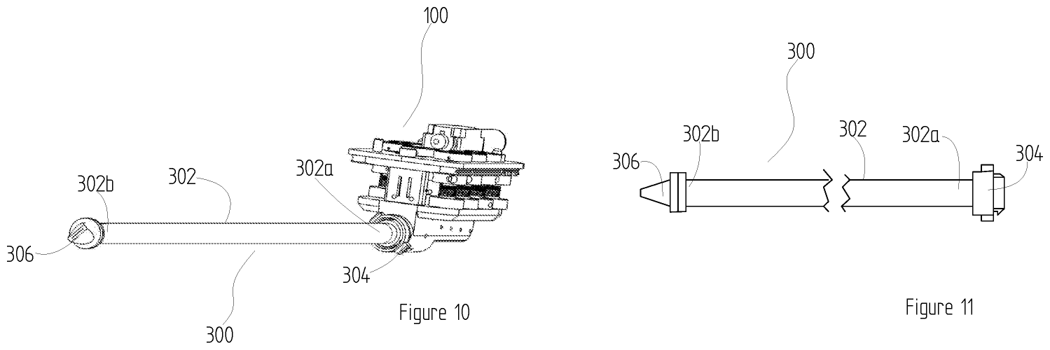

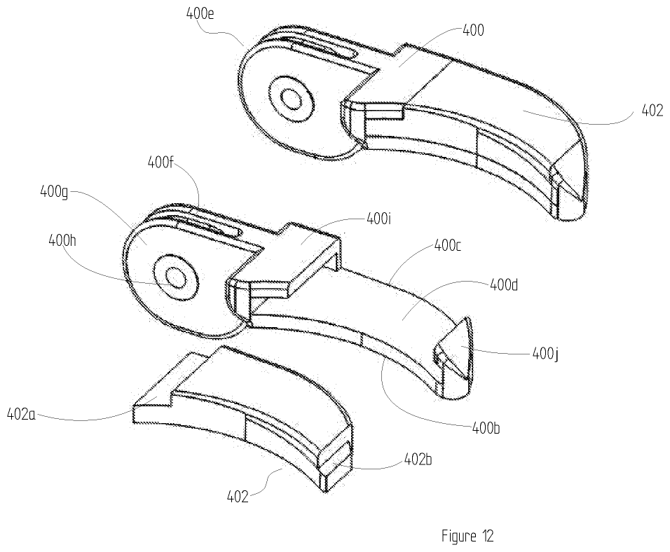

Referring to FIGS. 10 and 11, a protective sleeve (300) for use with surgical instruments (10) of embodiments of the invention is shown. The protective sleeve (300) comprises an elongate sheath (302) that has a first end (302a) and a second end (302b). The elongate sheath is formed from a thin plastic material and is flexible and compressible. The first end (302a) of the elongate sheath (302) is attachable to a surgical instrument by way of an attachment interface (304). The attachment interface may comprise a locking means such as a twist locking mechanism or snap fit interface or may be magnetic. The second end (302b) of the elongate sheath (302) defines an interface for attachment of an end closure (306) such as a duckbill valve or other type of suitable valve. The end closure (306) may be attached to the second end (302b) of the elongate sheath (302) by way of a locking means or magnetic attachment, for example.

In use, the end effector end of a surgical instrument (10) is inserted into the protective sleeve (300) after sterilisation. The protective sleeve (300) is attached to the surgical instrument (10) by way of the attachment interface (304). The surgical instrument (10) is inserted into a lumen of a port immediately prior to start of surgery. In embodiments utilising a magnet to attach the closure means (306) to the second end (302b) of the protective sleeve (300), the magnet is used to align the surgical instrument (10) with the lumen of the port. The closure means (306) is sized appropriately to enable it to extend through the lumen of the port. As the surgical instrument (10) is advanced, the surgical instrument (10) penetrates through the valve (306) and the protective sleeve (300) is compressed within the port to expose the surgical instrument (10).

Upon conclusion of surgery, the surgical instrument (100) is withdrawn from the patient and through the port into the protective sleeve (300). The surgical instrument passes back through the valve which closes once the surgical instrument is again fully enclosed by the protective sleeve (300). Prior to re-use, the surgical instrument is sterilised through autoclave, gas or radiation treatment and a new protective sleeve (300) is fitted to the surgical instrument (100). The used protective sleeve (300) is discarded as hazardous waste after surgery.

1. A surgical instrument comprising: a rigid shaft, at least one elbow joint hingedly coupled to the rigid shaft and a wrist joint coupled to the at least one elbow joint, wherein the wrist joint is configured to provide a first degree of freedom of movement and a second
degree of freedom of movement, wherein the second degree of freedom of movement is substantially perpendicular to the first degree of freedom of movement.

2-10. (canceled)

11. A surgical instrument according to claim 1, wherein the at least one elbow joint and the wrist joint are independently movable by way of tendon drive means.

12. A surgical instrument according to claim 1 further comprising an end effector.

13. A surgical instrument according to claim 12, wherein the end effector is a monopolar end effector or a bipolar end effector.

14. A surgical instrument according to claim 12, wherein the end effector is movable relative to the wrist joint by tendon drive means.

15. (canceled)

16. A surgical instrument comprising: a rigid shaft and at least one elbow joint hingedly coupled to the rigid shaft, wherein an end effector is coupled to the at least one elbow joint and wherein the rigid shaft and the at least one elbow joint define a continuous lumen therethrough, the continuous lumen receiving an auxiliary end effector or providing irrigation or suction functionality.

17. A surgical instrument according to claim 16, wherein the primary end effector is a cutting tool, grasping tool or cauterization tool.

18. A surgical instrument according to claim 16, wherein the auxiliary end effector is a fibre based laser, imaging probe or cauterization tool or provides suction or irrigation functionality.

19. A surgical instrument according to claim 16, wherein the auxiliary end effector is coupled to a flexible tube.

20. (canceled)

21. A surgical instrument comprising: a rigid shaft and at least one elbow joint coupled to the rigid shaft, wherein an end effector is coupled to the at least one elbow joint, said end effector being operable by tendons shrouded by Bowden cables to facilitate movement of said end effector relative to the at least one elbow joint.

22. (canceled)

23. A surgical instrument according to claim 21 further comprising a multi lumen insert positioned within the lumen of the at least one elbow joint, the multi-lumen insert comprising a plurality of lumens, wherein one or more of the plurality of lumens is configured to receive a respective tendon.

24. A surgical instrument comprising: a rigid shaft and at least two elbow joints coupled
to the rigid shaft by way of a mounting arrangement, wherein the mounting arrangement comprises a first part on one of said at least two elbow joints having a generally circular profile and a second part on a second of said at least two elbow joints comprising a generally triangular profiled groove for receiving the generally circular profile of the first part therein.

25. A surgical instrument according to claim 24, wherein the first part of the mounting arrangement is part of a planar apex defined by a chamfered elbow profile and wherein the second part of the mounting arrangement is part of a grooved apex defined by a chamfered elbow profile.

26. (canceled)

27. A surgical instrument according to claim 25, wherein the first part of the mounting arrangement and the second part of the mounting arrangement are co-operable to provide relative movement between a first of said at least two elbow joints and a second of said at least two elbow joints through an angular range of movement between sixty and one hundred degrees.

28. A device for laparoscopic surgery comprising:
a robotic arm comprising means for mounting the robotic arm to a surface and an end effector for mounting a port, wherein the end effector is movable relative to the means
for mounting the robotic arm to a surface through a plurality of selectively operable joints;
a port for mounting on the end effector of the robotic arm, wherein the port comprises a plurality of lumens for receiving respective surgical instruments; and
a surgical instrument for insertion within a lumen of the port, the surgical instrument comprising a rigid shaft, at least one elbow joint coupled to the rigid shaft and an end effector coupled to the at least one elbow joint.

29-44. (canceled)

45. A safety device for a robotic arm comprising a first operation switch and a second operation switch, wherein operation of the robotic arm is effected only by activation of both
the first operation switch and the second operation switch.

46. A safety device for a robotic arm according to claim 45, wherein the first operation switch and the second operation switch are provided on a handle for maneuvering the robotic arm.

47. A safety device for a robotic arm according to claim 45, wherein the first operation switch and the second operation switch are configured to be operated with the same hand.

48. A safety device for a robotic arm comprising position detecting means for determining the position of a surgical instrument in a defined workspace, the position detecting means being operably coupled to a lockout means, wherein upon detection of movement of the surgical tool outside of the defined workspace, the lockout means prevents further movement of the surgical tool.

49. A safety device for a robotic arm according to claim 48 wherein the position detecting means monitors the position of the robotic arm relative to a reference point and
records associated data.

50. A safety device for a robotic arm according to claim 49, wherein data recorded by the position detecting means is used to facilitate movement of the robotic arm from the point of lockout back to the reference point or other selected position using reverse movements to those recorded by the position detecting means.

51. A robotic arm comprising a plurality of electromagnetically braked joints and a position sensor associated with each electromagnetically braked joint, wherein each position sensor is operably connected to a processor, said processor monitoring the position of each electromagnetically braked joint relative to a pre-determined spatial threshold and locking each of said electromagnetically braked joints upon the processor detecting a signal from one or more position sensors signifying approach of one or more of said electromagnetically braked joints to a spatial threshold.

52. A robotic arm according to claim 51 further comprising an end effector mounting a surgical tool and a further position sensor associated with said end effector and operably connected to the processor, said processor monitoring the position of the end effector relative to the pre-determined spatial threshold and locking each of the electromagnetically braked joints upon the processor detecting a signal from the further position sensor signifying approach of the end effector to the spatial threshold.

53. A robotic arm according to claim 51 further comprising force detection means associated which each of the electromagnetically braked joints, wherein the force detection means determines a force direction relative to the spatial threshold and permits movement of each of the electromagnetically braked joints if it is determined that each electromagnetically braked joint and/or the end effector is being moved away from the spatial threshold and resists movement of each the electromagnetically braked joints and/or the end effector if it is determined that one or more of the electromagnetically braked joints and/or the end effector is being moved towards or across the spatial threshold.

54. A control system for a robotic surgical system comprising a plurality of motor controllers, a safety watchdog module, and a motherboard, wherein the safety watchdog and plurality of motor controllers are operably connected to the motherboard and wherein the safety watchdog module monitors at least one parameter of the robotic surgical system and is configured to isolate power from the motor controllers in response to detection by the safety watchdog module of one or parameters deviating from a pre-determined range or exceeding a pre-determined threshold.

55. A control system for a robotic surgical system according to claim 54, wherein the safety watchdog module and plurality of motor controller module are modular components
of the motherboard and can be selectively removed and replaced without removal of other modular components of the motherboard.

56-59. (canceled)
